(19) United States
(12) Patent Application Publication
(10) Pub. No.: US 2023/0255930 A1
(43) Pub. Date: Aug. 17, 2023

(54) METHOD OF INCREASING THE POPULATION OF COPROCOCCUS SPP. IN THE GUT MICROBIOME

(71) Applicant: DSM IP ASSETS B.V., Heerlen (NL)

(72) Inventors: Thanh-Van PHAM, Kaiseraugst (CH); Ateequr REHMAN, Kaiseraugst (CH); Nicole SEIFERT, Kaiseraugst (CH); Robert STEINERT, Kaiseraugst (CH); Wilbert SYBESMA, Kaiseraugst (CH)

(21) Appl. No.: 18/014,387

(22) PCT Filed: Jul. 8, 2021
(86) PCT No.: PCT/EP2021/068934

§ 371 Date: Jan. 4, 2023

(30) Foreign Application Priority Data
Jul. 9, 2020 (EP) 20184915.5

(51) Int. Cl. A61K 31/375; A61K 31/525; A61K 31/593; A61K 31/025; A61K 31/197; A61K 45/06; A61P 43/00

(52) U.S. Cl. CPC A61K 31/375; A61K 31/025; A61K 31/197; A61K 31/525; A61K 31/593; A61K 45/06; A61P 43/00

(57) ABSTRACT

The population of Coprococcus spp. bacteria in the gut microbiome is lower in people with various diseases. We have found that administration of vitamins / vitamin combinations such as : the combination of Vitamin B2 and C; Vitamin D3; beta-carotene, and Vitamin B5, when administered directly to the gut, so that it provides nourishment to the gut microbiome directly, can result in an increase of Coprococcus spp.

### BRIEF DESCRIPTION OF THE INVENTION

This invention relates to methods of increasing the gut microbiome population of Coprococcus spp., by delivering vitamins or combinations of vitamins directly to the gut of an animal, preferably a human. This can be accomplished by using, for example a delayed release formulation of the chosen vitamin or combination. Preferred vitamins include the combination of riboflavin and Vitamin C, vitamin D3, beta-carotene and vitamin B5.

### BACKGROUND OF THE INVENTION

Coprococcus is a genus of anaerobic bacteria which normally resides in the human gut, and includes various species, such as C. catus, C. comes, and C. eutactus.

Studies have shown that a lower than normal amount of Coprococcus spp. in the gut is associated with various seemingly unrelated disease states and/or adverse conditions. These include: Colorectal cancer; Autism spectrum disorder in children, including those with abdominal pain; Multiple Sclerosis; Obesity/ overweight status; Irritable Bowel Syndrome, Inflammatory Bowel Disease, both with or without accompanying depression; Generalized Anxiety Disorder, Depression, Migraine; Blood in stools (but absence of cancerous or pre-cancerous lesions); Ankylosing Spondylitis; Nonalcoholic Fatty Liver Disease (NALFD)/ Nonalcoholic steatohepatitis (NASH); Campylobacter infections; Preeclampsia; Constipation; Ulcerative colitis; Crohn’s Disease (and those receiving monoclonal antibody treatments for Chron’s disease); Coronary Heart Disease; Dermatitis and eczema; Parkinson’s Disease; Phenylketonuria (PKU); Epilepsy; Lowered Immunity; Neuromyelitis optica spectrum disorder; Allergic Disease in children; Collagenous colitis, Pervasive Developmental Disorder Not Otherwise Specified; and Chronic pancreatitis.

It would be desirable to provide a method to increase the population of Coprococcus in the gut microbiome, particularly in individuals which are experiencing or at risk on experiencing one of the aforementioned diseases/adverse conditions or symptoms associated with one of the diseases/adverse conditions.

### DETAILED DESCRIPTION ON THE INVENTION

A large number of studies have shown that the population of Coprococcus spp. in the gut microbiome is decreased when an animal, preferably a human is suffering from a particular disease/adverse condition as compared to the population present in the animal not suffering that particular disease/adverse condition. However, none of these studies have suggested a method of how to increase the population of Coprococcus spp., thus alleviating at least one of the symptoms of the disease/adverse condition. It has been found, in accordance with this invention, that direct delivery of certain vitamins or vitamin combinations to the large intestine of an animal, preferably a human, can provide nourishment to the gut microbiome, and increase the resident population of Coprococcus spp.

Thus, this invention relates to: methods of preventing, reducing the risk or delaying the onset of a disease or adverse condition; methods of treating a disease or adverse condition; and methods of meeting the nutritional needs of a person experiencing a disease or adverse condition, wherein the disease or adverse condition is characterized in a lower than normal population of Coprococcus spp. in the gut microbiome, by administering at least one vitamin or vitamin combination which is delivered directly into the large intestine.

Vitamins and vitamin combinations which have been found suitable for increasing the population of Coprococcus spp. in the gut comprise: a combination of riboflavin and vitamin C; vitamin D3; beta-carotene; and vitamin B5.

Thus one aspect of this invention is a method of increasing the population of Coprococcus spp. in the gut microbiome comprising administering a population-increasing effective amount of a vitamin selected from the group consisting of: a combination of riboflavin and vitamin C; vitamin D3, beta-carotene and vitamin B5 directly to the large intestine of an animal, preferably a human, in need thereof.

Another embodiment of this invention is the treatment and or prevention of a disease/adverse condition which is associated with a decreased population of Coprococcus spp. in the gut microbiome comprising administering a vitamin selected from the group consisting of: a combination of riboflavin and vitamin C; vitamin D3, beta-carotene, and vitamin B5 to an animal, preferably a human in need thereof, characterized in that the administration is directly to the large intestine of the animal.

Another embodiment of this invention is an oral delivery formulation comprising a Coprococcus spp. population increasing effective amount of a vitamin selected from the group of consisting of: a combination of riboflavin and vitamin C; vitamin D3, beta-carotene, and vitamin B5; and excipients, and said form is characterized in that the vitamin is delivered directly to the gut microbiome present in the large intestine.

### DEFINITIONS

As used throughout the specification and claims, the following definitions apply:

“Coprococcus spp.” means at least one species of the genus Coprococcus, and may include C. catus, C. comes, and/or C. eutactus.

“Decreased population” means that the amount of Coprococcus spp. present in the individual is lower compared to that found in a healthy population of people.

“Healthy” as used herein means the animal, including a human is not experiencing a disease/ adverse condition which is known to be associated with a decreased population of Coprococcus spp. in the gut microbiome.

The terms “Vitamin B2” and “riboflavin” are used interchangeably, and include their esters, and in particular riboflavin-5′-phosphate.

The term “Vitamin C” is used interchangeably with “ascorbic acid” and includes pharmaceutically acceptable salts thereof (e.g. sodium ascorbate and calcium ascorbate) and pharmaceutically acceptable esters thereof (in particular ascorbyl palmitate).

The term “Vitamin D” as used herein means vitamin D3. 25-hydroxyvitamin D3 can be use in lieu of or in addition to Vitamin D3, preferably in non-human species. The relative strength of 25-hydroxyvitamin D3 to Vitamin D3 is approximately 40:1, so dosing of 25-hydroxyvitamin D3 should be adjusted accordingly.

The term “Beta-Carotene” refers to β-carotene or Provitamin A.

The term “Vitamin B5” means pantothenic acid and pharmaceutically acceptable salts thereof (e.g. calcium pantothenate) and includes its derivate pantothenol or panthenol.

An animal, preferably a human “in need of having their population of Coprococcus spp. increased” is at risk of, or is currently experiencing at least one disease/ adverse condition selected from the group consisting of:
Colorectal cancer;
Autism spectrum disorder in children, including those with abdominal pain;
Multiple Sclerosis;
Obesity/ overweight status;
Irritable Bowel Syndrome;
Inflammatory Bowel Disease, both with or without accompanying depression;
Generalized Anxiety Disorder,
Depression,
Migraine;
Blood in stools (but absence of cancerous or pre-cancerous lesions);
Ankylosing Spondylitis;
Nonalcoholic Fatty Liver Disease (NALFD)/ Nonalcoholic steatohepatitis (NASH);
Campylobacter infections;
Preeclampsia;
Constipation;
Ulcerative colitis;
Crohn’s Disease (and those receiving monoclonal antibody treatments for Chron’s disease);
Coronary Heart Disease;
Dermatitis and eczema;
Parkinson’s Disease;
Phenylketonuria (PKU);
Epilepsy; Lowered Immunity;
Neuromyelitis optica spectrum disorder;
Allergic Disease in children;
Chronic pancreatitis;
Collagenous colitis; and
Pervasive Developmental Disorder Not Otherwise Specified.

“Prevention” is not limited to the state where a disease/adverse condition is never achieved. Instead, as used throughout the specification and claims, it can include lessening the severity of a disease/adverse condition, or a symptom thereof; delayed onset of a disease/adverse condition, or a symptom thereof; early intervention in a disease/adverse condition or symptom thereof; and lessening the risk of development of a disease/adverse condition, or symptom.

“Direct delivery” means that the vitamin and/or combination of vitamins is administered in a manner such that the vitamin and/or combination of vitamins is not absorbed in the stomach and/or small intestine; rather the vitamin and/or combination becomes present in the distal intestinal tract, preferably the large intestine, where it is available to the microbiome. These vitamins and/or combination are not part of a person’s usual daily nutritional requirements (generally obtained through diet and conventional vitamin supplementation), and are administered in excess thereof. For human use, the preferred method is through a form which delays delivery until the intestinal tract is reached. For non-human animals, a preferred delivery includes a method of administering a large enough dose so that only a portion of the vitamin delivered is absorbed in the stomach, and the remainder which is an effective dose, is available to the intestinal tract; although not preferred, this method of delivery can be used for humans as well.

### BRIEF DESCRIPTION OF THE FIGURES

FIG. 1. Relative abundance of Coprococcus upon administration of vitamins in in vitro experiment (A) and in human study (B). FIG. 1A shows log₁₀ fold changes of Coprococcus abundances in comparison to control in fermentation supernatant at 24h. FIG. 1B shows relative abundance of fecal Coprococcus upon administration of colon-targeted vitamins in the human study.

FIG. 2. Relative abundance of Coprococcus comes upon administration of colon-targeted vitamins in the human study.

### DISEASES/CONDITIONS/SYMPTOMS

Another embodiment of this invention is the use of a vitamin and/or vitamin combination formulated for direct delivery to the gut microbiome of an animal in the large intestine, preferably a human, and characterized in that upon delivery to the large intestine, it increases the population of Coprococcus spp. in the gut microbiome. Preferably the vitamin and/or vitamin combination is selected from the group consisting of: a combination of riboflavin and vitamin C; vitamin D3, beta-carotene and vitamin B5.

In some embodiments the population of Coprococcus spp. will be increased in a person at risk for or experiencing a disease or condition selected from the group consisting of: Colorectal cancer; Autism spectrum disorder in children, including those with abdominal pain; Multiple Sclerosis; Obesity/ overweight status; Irritable Bowel Syndrome; Inflammatory Bowel Disease, both with or without accompanying depression; Generalized Anxiety Disorder, Depression, Migraine; Blood in stools (but the absence of cancerous or pre-cancerous lesions); Ankylosing Spondylitis; Nonalcoholic Fatty Liver Disease (NALFD)/ Nonalcoholic steatohepatitis (NASH); Campylobacter infections; Preeclampsia; Constipation; Ulcerative colitis; Crohn’s Disease (and those receiving monoclonal antibody treatments for Crohn’s disease); Coronary Heart Disease; Dermatitis and eczema; Parkinson’s Disease; Phenylketonuria (PKU); Epilepsy; Lowered Immunity; Neuromyelitis optica spectrum disorder; Allergic Disease in children; Chronic pancreatitis; Collagenous colitis; and Pervasive Developmental Disorder Not Otherwise Specified; by administering a vitamin or vitamin combination selected from the group consisting of a combination of riboflavin and vitamin C, vitamin D3, beta-carotene and vitamin B5.

Another embodiment of this invention the non-therapeutic use of a vitamin and/or vitamin combination formulated for direct delivery to the gut microbiome and characterized in that upon delivery, it increases the population of Coprococcus spp. in the gut microbiome of an animal preferably a human. Preferably the vitamin and/or vitamin combination is selected from the group consisting of a combination of riboflavin and vitamin C, vitamin D3, beta-carotene and vitamin B5.

Another embodiment of this invention is the use of a vitamin and/or vitamin combination in the manufacture of a medicament formulated for direct delivery and characterized that upon delivery, it increases the population of Coprococcus spp. in the gut microbiome of an animal preferably a human. Preferably the vitamin and/or vitamin combination is selected from the group consisting of a combination of riboflavin and vitamin C, vitamin D3, beta-carotene and vitamin B5. In some embodiments the vitamin and/or vitamin combination will increase the population of Coprococcus spp. in an animal or person at risk for or experiencing a disease or condition selected from the group consisting of: Colorectal cancer; Autism spectrum disorder in children, including those with abdominal pain; Multiple Sclerosis; Obesity/ overweight status; Irritable Bowel Syndrome; Inflammatory Bowel Disease, both with or without accompanying depression; Generalized Anxiety Disorder, Depression, Migraine; Blood in stools (but absence of cancerous or pre-cancerous lesions); Ankylosing Spondylitis; Nonalcoholic Fatty Liver Disease (NALFD)/ Nonalcoholic steatohepatitis (NASH); Campylobacter infections; Preeclampsia; Constipation; Ulcerative colitis; Crohn’s Disease (and those receiving monoclonal antibody treatments for Crohn’s Disease); Coronary Heart Disease; Dermatitis and eczema; Parkinson’s Disease; Phenylketonuria (PKU); Epilepsy; Lowered Immunity; Neuromyelitis optica spectrum disorder; Allergic Disease in children; Chronic pancreatitis; Collagenous colitis; and Pervasive Developmental Disorder Not Otherwise Specified by administering a vitamin or vitamin combination selected from the group consisting of a combination of riboflavin and vitamin C, vitamin D3, beta-carotene and vitamin B5.

Another embodiment of this invention is a medical food for persons who have a disease which can benefit from an increase in Coprococcus spp. in their microbial biome. Thus another aspect of this invention is a vitamin and/or vitamin combination selected from the group consisting of: combination of riboflavin and vitamin C; vitamin D3, beta-carotene, and vitamin B5; and excipients; and said form is characterized in that the vitamin is delivered directly to the gut microbiome in the large intestine, used for addressing the nutritional needs of a patient experiencing a disease/adverse condition characterized by a lower than normal Coprococcus spp. population in the gut microbiome. Such diseases/adverse conditions include: Colorectal cancer; Autism spectrum disorder in children, including those with abdominal pain; Multiple Sclerosis; Obesity/ overweight status; Irritable Bowel Syndrome, Inflammatory Bowel Disease, both with or without accompanying depression; Generalized Anxiety Disorder, Depression, Migraine; Blood in stools (but absence of cancerous or pre-cancerous lesions); Ankylosing Spondylitis; Nonalcoholic Fatty Liver Disease (NALFD)/ Nonalcoholic steatohepatitis (NASH); Campylobacter infections; Preeclampsia; Constipation; Ulcerative colitis; Crohn’s Disease (and those receiving monoclonal antibody treatments for Crohn’s Disease); Coronary Heart Disease; Dermatitis and eczema; Parkinson’s Disease; Phenylketonuria (PKU); Epilepsy; Lowered Immunity; Neuromyelitis optica spectrum disorder; Allergic Disease in children; Chronic pancreatitis; Collagenous colitis; and Pervasive Developmental Disorder Not Otherwise Specified.

The aforementioned vitamins and combinations of vitamins may be administered as a sole active agents, or may be administered in combination with prebiotics, probiotics, other ingredients which modulate the gut microbiome, and conventional pharmaceutical or nutritional agents.

### Animals

“Animals” include mammals, poultry and preferably humans. Preferred non-human animals are companion animals, and include as dogs, cats, and horses. Among agriculturally important animals, preferred animals include poultry, swine, bovines, ovines and caprines and equines.

### Dosages

The dosages used herein are intended to be in addition to the active ingredients that is ingested for general nutrition purposes. Instead, they act upon the gut microbiome environment as a whole, at the genus, species and strain level of the gut microbes. The active agents are not intended to be metabolized directly by the animal, including the human. Rather they are intended to be utilized by the bacterial population of the colon. Therefore, the amounts reported below would be consumed by the animal in addition to the usual diet, but as they are not directly available to the animal due to their delayed release.

Suitable dosages per day are:

Beta Carotene: up to 150 mg per day. Preferably, β-carotene is administered in an amount such that its local concentration in the colon is at least 0.1 g/L, preferably at least 0.15 g/L, most preferably at least 0.2 g/L. Preferred local concentrations in the colon range from about 0.05 g/L to about 0.4 g/L, more preferably from about 0.15 g/L to about 0.25 g/L One preferred dosage per day is up to 150 mg. Other dosages can be about 5- 100 mg per day; 25- 85 mg per day, and 10-50 mg per day.

Vitamin B5: up to 1500 mg per day. Preferably, vitamin B5 is administered in an amount such that its local concentration in the colon is at least 1 g/L, preferably at least 1.5 g/L, most preferably at least 2 g/L. Preferred local concentrations in the colon range from about 0.5 g/L to about 4 g/L, more preferably from about 1.5 g/L to about 2.5 g/L One preferred dosage per day is up to 1500 mg.

Riboflavin: up to 200 mg per day; preferably 1-85 mg per day; more preferably 70-80 mg per day. Preferably, riboflavin is administered in an amount such that its local concentration in the colon is at least 0.05 g/L, preferably at least 0.1 g/L more preferably at 0.125 g/L. Preferred local concentrations in the colon range from about 0.1 g/L to about 0.5 g/L or from about 0.1 g/L to about 0.2 g/L, preferably about 0.125 g/L. One preferred dosage per day can be up to 200 mg.

Vitamin C: up to 2000 mg per day; preferably 400-600 mg per day; more preferably 450-550 mg per day. Preferably, ascorbic acid is administered in an amount such that its local concentration in the colon is at least 0.05 g/L, preferably at least 0.1 g/L, most preferably at least 0.8 g/L. Preferred local concentrations in the colon range from about 0.05 g/L to about 1.5 g/L, more preferably from about 0.5 g/L to about 1 g/L, most preferably from about 0.8 g/L to about 0.9 g/L. One preferred dosage per day is up to 2000 mg.

Vitamin D3: up to 250 micrograms per day; preferably 5-80 micrograms per day; more preferably 15-25 micrograms per day.

For formulations which deliver the vitamin/vitamin combination directly to the large intestine, dosages are preferably taken once per day, but may be taken in multiple smaller doses (i.e. two half-doses per day or three ⅓ does per day) if desired.

For dosages which are to be administered as a high dose rather than direct delivery to the large intestine, the amount may be at least about 10x or even 20x the recommended dose, for example if the recommended daily dose is 5 mg, the amount administered in the feed, food, or form is 50 mg or 100 mg in order for the vitamin or combination to be present in the colon.

It is preferred that the doses be taken for a sustained period of time, for example, at least one week, preferably at least 2 weeks, and more preferably at least one month. Doses may be taken for daily over a sustained period of time if desired.

### Formulations

A suitable formulation may include a high enough dosage so that a portion of the vitamin/ combination of vitamins is absorbed normally, but the remainder is available to the gut microbiome in the intestine at an effective amount. Other formulations include non-oral routes, such as via suppositories or injections. Preferred formulations are delayed release oral formulations.

A used herein, “delayed release” refers to the release of the active agent at a time later than immediately after administration. Preferably, “delayed release” means delivery of the active agent, upon oral administration, to the large intestine, preferably the colon, in a delayed manner relative to an immediate release formulation.

An “enteric layer” is a layer surrounding a core, wherein the core comprises the active agent and the layer confers resistance to gastric juice. An “enteric shell” is a shell or matrix surrounding or encapsulating the active agent, wherein the shell confers resistance to gastric juice. Alternatively, a matrix-based delivery system can be used. Matrix based systems have no discrete layer of coating material but the active agent is more or less homogeneously distributed within the matrix. Further, there are colon-release systems that embed the active agent in e.g. in a fiber matrix (enzyme-triggered) and an enteric coating on top.

In a preferred embodiment for humans, the formulation of the present invention is a solid dosage form for oral administration. The formulation may be in the form of a capsule, pellet, bead, sphere, mini spheres, tablet, mini tablet, or granule, optionally coated with a delayed release coating or shell that prevents the release of the active agent before the small intestine, preferably before the colon.

Coating, shell, or matrix materials for the delayed release of the active agent, in particular for targeted release in the ileum or the large intestine, upon oral administration are known in the art. They can be subdivided into coating materials that disintegrate above a specific pH, coating materials that disintegrate after a specific residence time in the gastrointestinal tract and coating materials that disintegrate due enzymatic triggers specific to the microflora of a specific region of the intestines. Coating or shell materials from different categories are commonly used in combinations. Coating or shell materials of these three different categories for targeting to the large intestine have been reviewed for example in Bansal et al. (Polim. Med. 2014, 44, 2,109-118). In one embodiment of the present invention the delayed release coating comprises at least one component selected from coating materials that disintegrate pH-dependently, coating materials that disintegrate time-dependently, coating materials that disintegrate due to enzymatic triggers in the intestinal environment (e.g. in the intestinal environment of the ileum and the large intestine), and combinations thereof.

Coating materials that disintegrate pH-dependently include polyvinyl acetate phthalate, cellulose acetate trimellitate, hydroxypropyl methylcellulose phthalate HP-50, HP-55 or HP-55S, cellulose acetate phthalate, shellac, hydroxypropyl methylcellulose acetate succinate (HPMCAS), poly(methacrylic acid, ethyl acrylate) 1:1 (Eudragit® L100-55, Eudragit® L30D-55), poly(methacrylic acid, methyl methacrylate) 1:1 (Eudragit® L-100, Eudragit® L12.5), poly(methacrylic acid, methyl methacrylate) 1:2 (Eudragit® S-100, Eudragit® S12,5, and Eudragit® FS30D).

Coating materials that disintegrate time-dependently include Eudragit® RL, Eudragit®RS, and ethylcellulose.

Coating materials that disintegrate due to enzymatic triggers in the large intestinal environment include chondroitin sulfate, pectin, guar gum, chitosan, inulin, lactulose, raffinose, stachyose, alginate, dextran, xanthan gum, locust bean gum, arabinogalactan, cyclodextrin, pullulan, carrageenan, scleroglucan, chitin, curdulan, levan, amylopectin, starch, amylose, resistant starch, and azo compounds being degraded by azo bonds splitting bacteria.

In one embodiment the formulation comprises an enteric capsule, filled with a composition comprising the active agent. The enteric capsule confers resistance against the acidic environment of the stomach. For example, softgel formulations may deliver the active agent in solution and yet offer advantages of solid dosage forms. Softgel capsules are particularly suited for hydrophobic active agents which do not dissolve readily in water. Vitamin K and omega-3 fatty acids are preferably formulated in softgel capsules.

In another embodiment, the formulation is a tablet comprising (i) a core comprising the active agent, and (ii) a delayed release coating such as an enteric coating. This may be a hard gel capsule.

The release of the active agent(s) may be delayed until small intestine. In another embodiment, the release of the active agent(s) is delayed until the distal small intestine. In yet another embodiment, the release of the active agent(s) is delayed until the colon.

The following non-limiting Examples better illustrate the invention.

### EXAMPLES

### Example 1

### In Vitro Fermentation Study

### Donor and Sample Preparation

At the start of this intestinal batch fermentation incubation, all test ingredients were added from stock solutions to the modified nutritional medium, containing (g/l): 2.5 K2HPO4, 10.9 KH2PO4, 2 NaHCO3, 2 yeast extract, 2 peptone, 1 mucin, 0.5 cysteine, 2 Tween 80, 2 glucose, 2 starch, 2 cellobiose, 0.1 NaCl, 0.01 MgSO4.7H2O, 0.01 CaCl2.6H2O, 0.05 hemin, 0.5 bile salts.

The following compounds were added from stock solutions prepared in water:
Beta-carotene,
Vitamin B3,
Vitamin B5,
Vitamin B7,
Fructooligosaccharides Fructooligosaccharides are included as a positive control.

Each compound was tested in three different concentrations; an overview is given in Table 1, below.

TABLE 1

Dose designation and final concentration of micronutrients in in vitro fermentation experiment

| | | |
|---|---|---|
| | Dose | Final concentration |
| Beta-carotene | 0.2x | 0.048 mg/ml |
| | 1x | 0.24 mg/ml |
| | 5x | 1.2 mg/ml |
| Vitamin B3 (Niacinamide) | 0.2x | 0.002625 mg/ml |
| | 1x | 0.013125 mg/ml |
| | 5x | 0.065625 mg/ml |
| Vitamin B5 (Pantothenic acid) | 0.2x | 0.45 mg/ml |
| | 1x | 2.25 mg/ml |
| | 5x | 11.25 mg/ml |
| Vitamin B7 (Biotin) | 0.2x | 5 mg/ml |
| | 1x | 25 mg/ml |
| | 5x | 125 mg/ml |
| Fructooligosaccharide (FOS) | 0.5x | 0.5 mg/ml |
| | 1x | 1 mg/ml |
| | 2x | 2 mg/ml |

As a source of the microbial community, freshly prepared fecal suspension from a human donor was added to the reactors. Each reactor had a volume of 70 ml. All tests, except the blanks, were performed in single repetition. Incubation conditions were 48 h at 37° C., under shaking (90 rpm) and anaerobic conditions.

### Measurements

Microbial composition: Illumina sequencing was performed at the start and after 24 h of incubation. The technique targets the16S rRNA gene that consists of variable and conserved regions, spread over the gene. Due to their key role in protein expression, the conserved regions are characterized by very low evolutionary rates.

The methodology applied involves primers that span 2 hypervariable regions (V3-V4) of the 16S rRNA gene. Using a pair-end sequencing approach, sequencing of 2×250 bp results in 424 bp amplicons. Such fragments are taxonomically more informative as compared to smaller fragments. In brief, mothur (v. 1.42.0) was used to assemble reads into contigs, perform alignment-based quality filtering (alignment to the mothur-reconstructed SILVA SEED alignment, v. 123), remove chimeras, assign taxonomy using a naïve Bayesian classifier and SILVA NR v132 and cluster contigs into OTUs. All sequences classified as Eukaryota, Archaea, Chloroplasts and Mitochondria were removed, as well as sequences that could not be classified. For each OTU, representative sequences were selected as the most abundant sequence within that OTU.

### Example 2

### Clinical Study

### Human Subjects

Twelve participants were allocated to each of the six vitamin groups, and 24 participants allocated to the placebo group. All 96 participants completed the intervention. To be considered eligible for enrolment into the study, participants have to be able to give written informed consent; be aged between 20 and 50 years of age; have a BMI of between 18.5 - 30 Kg/m2; have a stable body weight (< 5% change) over the past 3-months; be in generally good health, as determined by the investigator; have not consumed dietary supplements, prebiotic, probiotic, dietary or fiber-rich supplements within 4 weeks prior to baseline visit and be willing to avoid these supplements until the end of the study; be willing to avoid liver consumption for the duration for the study, be willing to maintain their current level of physical activity for the duration of the study; and be willing to consume the IP daily for the duration of the study. Any participants who have a typical fiber intake >30 g/day; were pregnant or planning to become pregnant, have consumed disallowed medications; had made major dietary changes over the past three months or had planned major lifestyle changes; had taken part in a study within the previous 60 days; or had any ongoing or previous illness that the investigator deemed would impact on the objectives of the study were excluded. The study protocol was approved by the Clinical Research Ethics Committee of the Cork Teaching Hospitals (Protocol Number: AFCRO-087) and performed in accordance with the Declaration of Helsinki. Each subject provided written informed consent before inclusion in the study. The trial was registered with clinicaltrials.gov under the ID: NCT03668964.

### Study Design

The trial was a randomized, double-blind, placebo-controlled, parallel study in which subjects received either the vitamin supplement or placebo over four weeks. There were three visits: 1) screening; 2) baseline (one week after screening) and 3) follow-up (four weeks after baseline). At the screening visit (Visit 1), informed consent was obtained, and eligibility was reviewed including a medical history interview and a physical exam. Eligible participants started a one-week run-in period and were instructed to refrain from extreme diets. The participants completed an eDiary daily and collected a fecal sample in the 48 hours prior to their randomization visit. Before the randomization visit, participants food frequency questionnaires were analyzed to ensure their typical fiber intake is <30 g fiber/day. Any participants outside this criterion, or outside any of the other eligibly criteria were excluded.

At the baseline visit (Visit 2), participants retuned a fecal samples collected in the previous 48 hours and eligibility was assessed. Eligible participants were enrolled and allocated a randomization number, and a 4-week supply of investigational product (IP) from one of the seven arms. Both the participant and research staff were blinded to the allocation. Participants completed the GSRS the SF-36 questionnaires. A bloods sample was collected and stored onsite at -80° C. Participants were instructed refrain from extreme diets, complete their eDiary daily, and to consume one capsule daily for the next 4 weeks.

At the final visit (Visit 3) participants retuned a fecal samples collected in the previous 48 hours. Participants completed the GSRS the SF-36 questionnaires. A bloods sample was collected and stored onsite at -80° C. Participants returned their IP and compliance was assessed.

### Investigational Products

Investigational products were as follows:
1) vitamin A (250 µg retinol equivalents (RE)/day),
2) vitamin C (500 mg ascorbic acid/day),
3) vitamin B2 (75 mg/day) + vitamin C (500 mg/day),
4) vitamin D3 (60 µg cholecalciferol /day), or
5) 200 mg/day microcrystalline cellulose (placebo).

All vitamins were provided by DSM Nutritional Products Ltd (Kaiseraugst, Switzerland); placebo was obtained from Fagron (Waregem, Belgium). Investigational products were formulated as a colon-release form in hard gelatin capsules (Lonza, Bornem, Belgium) coated using the pH-dependent polymer Eudragit S100 (Evonik Nutrition & Care GmbH, Darmstadt, Germany) that has been validated for targeted colon delivery (Cole et al., 2002). The selected doses were based on high dose oral delivery of vitamins in previous studies (de Vries et al., 2006; Lakoff et al., 2014; Cantarel et al., 2015; Steinert et al., 2016; Tang et al., 2016) subtracting estimated intestinal absorption level for each vitamin (Graf, 1980; Basu and Donaldson, 2003; Gropper et al., 2004; Reboul, 2013). All doses were below the upper limits published by EFSA, except vitamin B2 with no upper limit established (https://www.efsa.europa.eu/sites/default/files/assets/UL Summary tables.pdf).

### Measurements

Fecal microbial composition: Total DNA was extracted from all fecal samples collected throughout the study using the QlAamp DNA stool minikit (Qiagen, Crawley, United Kingdom) according to the manufacturer’s instructions, apart from addition of a bead-beating step and increasing the lysis temperature to 95° C. as described previously. After DNA isolation, DNA was quantified using the Qubit High Sensitivity DNA assay (Thermo Fisher). Whole metagenome libraries were then prepared using the Illumina Nextera XT kit (Illumina) in accordance with the manufacturer’s instructions, with the following modifications: Firstly, tagmentation time was increased to 7 min and secondly, following incorporation of indices and Ampure purification of the products, the samples were each individually sized by running on an Agilent High Sensitivity Chip (Agilent) and quantified using the Qubit High Sensitivity DNA assay (Thermo Fisher) in accordance with Teagasc Sequencing Platform SOPs. The samples were then pooled equimolarly and sequenced on the Illumina NextSeq 500 with a NextSeq 500/550 v2 high-output reagent kit (300 cycles). All sequencing was done in the Teagasc sequencing facility in accordance with standard Illumina sequencing protocols. Delivered raw FASTQ sequence files were quality checked as follows: poor quality and duplicate read removal, as well as trimming was implemented using a combination of SAM and Picard tools. Taxonomy was assigned to the reads using the Metaphlan2 software.

### Results

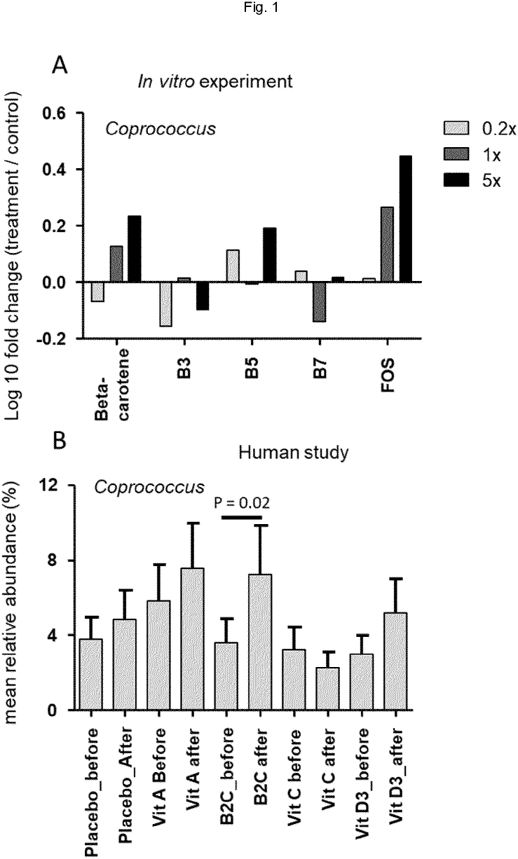

### Relative Abundance of the Genus Coprococcus and the Species Coprococcus Comes (FIG. 1)

### In Vitro Experiment (FIG. 1A)

There was no distinct increase in Coprococcus relative abundances with vitamin B3 and B7.In contrast, administration of beta-carotene at 1x and 5x and vitamin B5 at 0.2x and 5x increased Coprococcus relative abundance. This increase was comparable to what was observed with the prebiotic FOS.

### Human Study (FIG. 1B)

Colon-targeted delivery of a combination of vitamin B2 and C for four weeks led to a significant (p = 0.02) increase in relative abundance of Coprococcus compared to baseline. There were additional trends for an increase in Coprococcus in response to vitamin A, and D3, but not vitamin C, however, this was not significant.

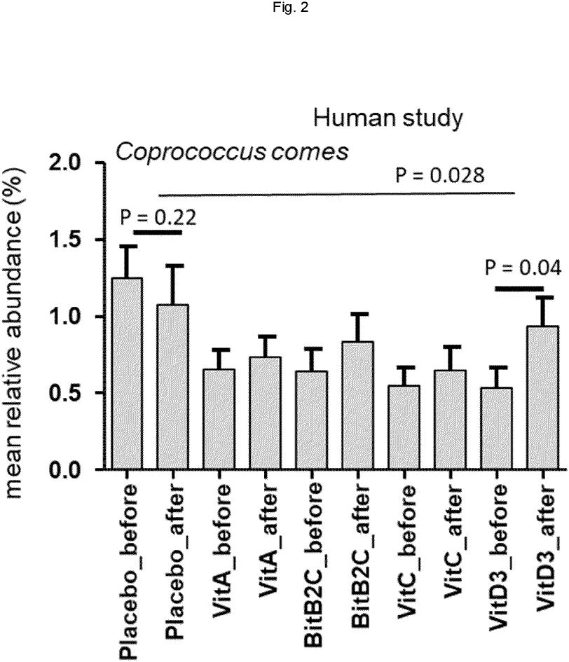

### Human Study (FIG. 2)

Colon-targeted delivery of vitamin D3 for four weeks led to a significant increase in Coprococcus comes relative abundance when compared to baseline (p = 0.04) and when compared to placebo group (p = 0.028).

1. Use of a vitamin or vitamin combination selected from the group consisting of:
a combination of riboflavin and vitamin C,
vitamin D3,
beta-carotene, and
vitamin B5,
which is formulated for direct delivery to the gut microbiome of an animal, preferably a human, wherein upon delivery, it increases the population of Coprococcus spp. in the gut microbiome.

2. The use according to claim 1 wherein the human is a person at risk for or experiencing a disease or adverse condition selected from the group consisting of: Colorectal cancer; Autism spectrum disorder in children, including those with abdominal pain; Multiple Sclerosis; Obesity/ overweight status; Irritable Bowel Syndrome, Inflammatory Bowel Disease, both with or without accompanying depression; Generalized Anxiety Disorder, Depression, Migraine; Blood in stools (but the absence of cancerous or pre-cancerous lesions); Ankylosing Spondylitis; Nonalcoholic Fatty Liver Disease (NALFD)/ Nonalcoholic steatohepatitis (NASH); Campylobacter infections; Preeclampsia; Constipation; Ulcerative colitis; Crohn’s Disease (and those receiving monoclonal antibody treatments for Crohn’s disease); Coronary Heart Disease; Dermatitis and eczema; Parkinson’s Disease; Phenylketonuria (PKU); Epilepsy; Lowered Immunity; Neuromyelitis optica spectrum disorder; Allergic Disease in children; Chronic pancreatitis; Collagenous colitis; and Pervasive Developmental Disorder Not Otherwise Specified.

3. The use according to claim 1 in combination with at least one of: prebiotics, probiotics, other ingredients which modulate the gut microbiome, and conventional pharmaceutical or nutritional agents.

4. A method of increasing the population of Coprococcus spp. in the gut microbiome comprising: administering a population-increasing effective amount of a vitamin selected from the group consisting of:
a combination of riboflavin and vitamin C,
vitamin D3,
beta-carotene, and
vitamin B5o an animal, preferably a human in need thereof.

5. A method according to claim 4 wherein the animal is a human and is at risk for or experiencing a disease or condition selected from the group consisting of: Colorectal cancer; Autism spectrum disorder in children, including those with abdominal pain; Multiple Sclerosis; Obesity/ overweight status; Irritable Bowel Syndrome, Inflammatory Bowel Disease, both with or without accompanying depression; Generalized Anxiety Disorder, Depression, Migraine; Blood in stools (but the absence of cancerous or pre-cancerous lesions); Ankylosing Spondylitis; Nonalcoholic Fatty Liver Disease (NALFD)/ Nonalcoholic steatohepatitis (NASH); Campylobacter infections; Preeclampsia; Constipation; Ulcerative colitis; Crohn’s Disease (and those receiving monoclonal antibody treatments for Crohn’s disease); Coronary Heart Disease; Dermatitis and eczema; Parkinson’s Disease; Phenylketonuria (PKU); Epilepsy; Lowered Immunity; Neuromyelitis optica spectrum disorder; Allergic Disease in children; Chronic pancreatitis; Collagenous colitis; and Pervasive Developmental Disorder Not Otherwise Specified.

6. A method according to claim 5, further comprising: administering at least one of: prebiotics, probiotics, other ingredients which modulate the gut microbiome, and conventional pharmaceutical or nutritional agents.

7. A vitamin or vitamin combination selected from the group consisting of:
a combination of riboflavin and vitamin C,
vitamin D3,
beta-carotene, and
vitamin B5;
and excipients
and said form wherein the vitamin is delivered directly to the large intestine, used for addressing the nutritional needs of a patient experiencing a disease/adverse condition characterized by a lower than normal Coprococcus spp. population in the gut microbiome.

8. A vitamin or vitamin combination according to claim 7, wherein the disease/adverse condition is selected from the group consisting of: Colorectal cancer; Autism spectrum disorder in children, including those with abdominal pain; Multiple Sclerosis; Obesity/ overweight status; Irritable Bowel Syndrome, Inflammatory Bowel Disease, both with or without accompanying depression; Generalized Anxiety Disorder, Depression, Migraine; Blood in stools (but the absence of cancerous or pre-cancerous lesions); Ankylosing Spondylitis; Nonalcoholic Fatty Liver Disease (NALFD)/ Nonalcoholic steatohepatitis (NASH); Campylobacter infections; Preeclampsia; Constipation; Ulcerative colitis; Crohn’s Disease (and those receiving monoclonal antibody treatments for Crohn’s disease); Coronary Heart Disease; Dermatitis and eczema; Parkinson’s Disease; Phenylketonuria (PKU); Epilepsy; Lowered Immunity; Neuromyelitis optica spectrum disorder; Allergic Disease in children; Chronic pancreatitis; Collagenous colitis; and Pervasive Developmental Disorder Not Otherwise Specified.
